(19) United States
(12) Patent Application Publication
(10) Pub. No.: US 2024/0260982 A1
(43) Pub. Date: Aug. 8, 2024

(54) INTRAVASCULAR LITHOTRIPSY CATHETER WITH MOVABLE EMITTERS

(71) Applicant: Shockwave Medical, Inc., Santa Clara, CA (US)

(72) Inventor: Susan PETERSON, Palo Alto, CA (US)

(73) Assignee: Shockwave Medical, Inc., Santa Clara, CA (US)

(21) Appl. No.: 18/428,976

(22) Filed: Jan. 31, 2024

Related U.S. Application Data
(60) Provisional application No. 63/442,980, filed on Feb. 2, 2023.

(51) Int. Cl. A61B 17/22; A61B 17/00

(52) U.S. Cl. CPC A61B 17/22022; A61B 2017/00017; A61B 2017/00292; A61B 2017/00778; A61B 2017/22001; A61B 2017/22025

(57) ABSTRACT

A catheter for treating an occlusion in a body lumen includes an elongate tube extending in a longitudinal direction from a distal region to a proximal region; a flexible enclosure at least partially secured to the distal region of the elongate tube; a first shock wave emitter located along the elongate tube; and a second shock wave emitter located along the elongate tube and translatable in the longitudinal direction relative to the first shock wave emitter.

### PRIORITY

This application claims priority to U.S. Provisional Patent Application Ser. No. 63/442,980, filed Feb. 2, 2023, the entire disclosure of which is incorporated by reference. This application is related to U.S. patent application Ser. No. 18/428,752, entitled “INTRAVASCULAR LITHOTRIPSY CATHETER WITH MOVABLE EMITTERS,” filed on Jan. 31, 2024, the entire disclosure of which is incorporated by reference.

### FIELD OF THE DISCLOSURE

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than ten atmospheres), causing the balloon to expand in a vessel to push calcified plaques back into the vessel wall and dilate occluded regions of vasculature. However, traditional angioplasty balloons are not always successful at dilating calcified lesions or vasculature, due to the stiffness and hardness of the calcified tissue and/or plaque.

More recently, catheters have been developed that include shock wave emitters (e.g., one or more electrode pairs) for generating shock waves inside an angioplasty balloon-this treatment is referred to as intravascular lithotripsy (IVL), a technology pioneered by Shockwave Medical, Inc., the applicant for the present application. Shock wave devices can be particularly effective for treating calcified lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with the calcified lesion in a body lumen. The balloon is then inflated with conductive fluid (using a relatively low pressure of two to four atmospheres) so that the balloon expands to contact the lesion, and thereby apposition the shock wave emitters proximate to the lesion. The shock wave emitters can then be activated to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the vessel.

For electrohydraulic generation of acoustic shock waves, a conductive solution (e.g., saline) may be contained within an enclosure that surrounds electrodes or may be flushed through a lumen to the electrodes. The calcified plaque modification is achieved by creating acoustic shock waves within the catheter by an electrical discharge across the electrodes. The energy released by the electrical discharge enters the surrounding fluid faster than the speed of sound, generating an acoustic shock wave. The energy from the electrical discharge may also create a rapidly expanding and collapsing vapor bubble that results in an additional shock wave. These shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble and acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

Traditional treatments for calcified lesions in patient vasculature often include mechanical alterations to the blood vessels, particularly atherectomy surgical procedures, which carry relatively greater risks of embolization (dislodgement of debris), abrupt closure (vessel collapse), dissection (tearing of vessel tissue), and/or perforation (puncturing of vessel tissue). The use of IVL in lieu of or in combination with traditional treatments for calcified lesions can greatly reduce the risk of embolization and perforation while concurrently leading to improved therapeutic outcomes in removing the calcified lesions.

Building on these advances, currently available shock wave catheters are limited by the length of the balloon and the position of emitters within that balloon. In patients where lesions are longer than the standard length of balloon, a physician may elect to proceed with repeated cycles of inflation, emitting shock waves, deflation, and readjustment of balloon position in order to deliver shock wave treatment to the complete length of a given lesion. When used over repeated cycles, there is concern about the structural integrity of the balloon, and whether the balloon can retain its deflated profile for crossing a lesion. Further, any procedure or technique that adds time to a patient being on the table longer than needed is not well received in clinical cases because it can cause procedural complications.

Thus, longer balloons are desired by physicians to treat longer lesions to reduce procedure time and costs. Simply using a longer balloon may not suffice, as the distance from the emitters to relatively distant parts of a longer balloon may lead to insufficient shock wave strength at the extremities of the balloon. Further, it may not be practical or possible to simply increase the number of shock wave emitters as adding emitters tends to increase a catheter's profile and complicate its design. Such a catheter would also require a substantially higher energy output from a power supply. A high number of emitters can also reduce the flexibility and trackability of the balloon region of the catheter, limiting the treatment sites which can be reached.

Accordingly, there is an unmet need for catheter and emitter assembly designs capable of delivering shock waves for relatively long lesions within a patient, and accordingly minimizing the time a patient is in surgery receiving treatments.

### BRIEF SUMMARY

The above objects are realized in a catheter that includes a set of movable emitters that can be repositioned while deployed within a patient. More specifically, the innovation of present disclosure allows the end user (e.g., a physician) to physically move the emitters of an IVL device along the catheter inside relatively long balloons and position those emitters in various places alongside a long, calcified lesion as desired in order to disrupt calcium with shock wave technology.

Various examples can allow the user to keep very long balloons (e.g., balloons having working lengths 100 mm-300 mm) inflated and in position in long lesions and instead move any number of emitters to desired location along the calcified lesion length inside the balloon that is inflated. This process can also be accomplished with the balloon slightly inflated, or semi-inflated depending on the calcium structure of the artery and there is no need to reposition the balloon at all.

In one or more embodiments, a catheter for treating an occlusion in a body lumen includes an IVL balloon with its emitters mounted on a movable tube.

In one or more examples, a catheter is configured for in situ customizable placement of emitters along a balloon length so that shockwave therapy can be applied optimally with minimal repositioning of the balloon required by the physician. Various embodiments of a catheter allow for efficient application of sonic energy to calcified regions of a vessel and avoids indiscriminately applying shock wave therapy to non-calcified regions of the vessel. Various embodiments also allow longer IVL balloons to be developed with relatively lower power requirements. Various embodiments of a catheter may improve flexibility, trackability, and/or deliverability of the catheter overall.

In one or more embodiments, an IVL catheter includes one or more shock wave emitters within an inflated IVL balloon that can be pushed distally or pulled proximally within the balloon along its working length to locations identified by a physician as needing shock wave treatment. In some embodiments, the emitters may be repositioned individually. In some embodiments, the emitters may be repositioned in small numbers (e.g., pairs). In some embodiments, the emitters may be repositioned completely in a connected assembly. Longtudinal adjustability may allow a relatively smaller number of emitters (e.g., one, two, three, four, or five emitters) to treat a long lesion of a body lumen and provide optimized energy delivery to target regions rather than a blanket dose over the whole length of the balloon whether or not calcium is present at each emitter location. In some embodiments, safety stops are incorporated to prevent the emitters from being placed too close to each other.

In one or more embodiments, a large number of shock wave emitters (e.g., six, seven, eight, nine, or more) may be placed along the length of a balloon, and an elongate member having a conducting region may be translatably located within the balloon to electrically connect select shock wave emitters to a power source. In some embodiments, the elongate member may be connected at its proximal end to a handle for moving the elongate member in the distal and proximal direction. Such embodiments may result in a lower profile catheter because fewer wires may be necessary for electrically connecting each individual emitter to the power source, while still allowing in situ customization for the physician. Such embodiments may also advantageously be used with a power source with a lower power output than one that may be normally necessary to generate shock waves at the large number of shock wave emitters.

In one or more embodiments, a shock wave catheter may include a large number of emitters (e.g., twelve or more) located in a balloon and configured to generate shock waves at only select emitters. In some embodiments, a shock wave catheter may be limited to a smaller number of total emitters (e.g., 8) that can be fired each cycle through input at the connector cable or generator so a physician would select the most critical locations along the balloon length and only those regions would receive energy during that cycle. This approach would allow longer balloons within the current generator output, and allow physician optimization of the therapy location.

Various examples described herein allow the end user to physically move the IVL emitters inside very long balloons and position them in various places in a long-calcified lesion as desired in order to disrupt the calcium.

According to an aspect of the disclosure, a catheter for treating an occlusion in a body lumen includes: an elongate tube extending in a longitudinal direction from a distal region to a proximal region; a flexible enclosure at least partially secured to the distal region of the elongate tube; a first shock wave emitter located along the central tube; and a second shock wave emitter located along the central tube and translatable in the longitudinal direction relative to the first shock wave emitter.

The flexible enclosure may have a working length d, and a center-to-center distance between the first shock wave emitter and the second shock wave emitter may be adjustable between 2 mm and d.

The first shock wave emitter may include a first pair of shock wave emitters and the second shock wave emitter may include a second pair of shock wave emitters that is translatable as a pair relative to the first pair.

The first shock wave emitter may include a first plurality of shock wave emitters and the second shock wave emitter may include a second plurality of shock wave emitters that is translatable as a group relative to the first plurality of emitters.

The catheter may include a third shock wave emitter that is independently translatable relative to the first and second shock wave emitters.

The catheter may include a safety stop fixedly located on the elongate tube and configured to space the first and second shock wave emitters by a center to center distance of no less than 2 mm.

The first shock wave emitter may be translatable in the longitudinal direction (i.e., in a proximal-distal direction).

The catheter may include a proximal handle that is configured to control the movement of the emitter assembly.

The proximal handle may include a first thumbwheel ratchet for controlling movement of the first shock wave emitter and a second thumbwheel ratchet for controlling movement of the second shock wave emitter.

The flexible enclosure may be an angioplasty balloon having a working length of at least 50 mm.

Each of the first and second shock wave emitters may include one or more electrode pairs and each of the one or more electrode pairs may include an outer electrode and an inner electrode.

According to an aspect of the disclosure, a catheter for treating a lesion in a body lumen may include: an elongate tube extending in a longitudinal direction from a distal region to a proximal region; an enclosure secured circumferentially around at least a portion of the distal region of the elongate tube; a proximal emitter assembly fixedly located on the central tube and inside of the enclosure; a distal emitter assembly fixedly located on the central tube and inside of the enclosure; and a longitudinally translatable elongate member having a distal region movably located along the central lumen, the elongate member configured to, in a proximal configuration, supply power to the proximal emitter assembly and, in a distal configuration, supply power to the distal emitter assembly.

The proximal emitter assembly may include one or more electrically connected proximal electrode pairs including a first proximal electrode that is electrically connected to a power supply, and, in the proximal configuration, the longitudinally translatable energy guide may be electrically connected to a second proximal electrode of the proximal electrode pairs such that, when a voltage pulse is applied from the power supply to the proximal emitter assembly, each of the one or more proximal electrode pairs generates a shock wave.

The distal emitter assembly may include one or more electrically connected distal electrode pairs including a first distal electrode that is electrically connected to the power supply, and, in the distal configuration, the longitudinally translatable power member may be electrically connected to a second distal electrode of the distal electrode pairs such that, when a voltage pulse is applied from the power supply to the distal emitter assembly, each of the one or more distal electrode pairs generates a shock wave.

The one or more proximal electrode pairs and one or more distal electrode pairs each may include an inner electrode and an outer electrode made of a conductive sheath.

In the proximal configuration, shock waves may be generated at the proximal emitter assembly and not at the distal emitter assembly, and, in the distal configuration, shock waves may be generated at the distal emitter assembly and not at the proximal emitter assembly.

The distal region of the translatable elongate member may include a radiopaque marker.

The catheter may include a proximal handle to control the movement of the elongate member between the proximal configuration to the distal configuration.

According to an aspect of the disclosure, a method for treating an occlusion in a body lumen includes: providing a catheter including a central tube extending from a proximal region to a distal region and defining a longitudinal direction and having a central lumen, an enclosure sealed to and surrounding at least a portion of a distal region of the central tube, and a shock wave emitter assembly including a first shock wave emitter and a second shock wave emitter located along the central tube within the enclosure, the first and second shock wave emitters movable in the longitudinal direction relative to the other; inserting the catheter into the body lumen and positioning the enclosure adjacent to the occlusion; filling the enclosure with a conductive fluid and anchoring the enclosure to a wall of the body lumen; moving the shock wave emitter assembly 1 mm or less away in the longitudinal direction from the blockage or restriction; and generating one or more shock waves from the at least one shock wave source.

The method may include imaging the body lumen with one or more of x-ray fluorescence, intravascular ultrasound, and optical coherence tomography, wherein the shock wave emitter assembly includes an imaging marker.

Each shock wave emitter may include an electrode pair and generating the one or more shock waves comprises applying a high voltage pulse from a power source.

The high voltage pulse may include a voltage between 1 kV and 15 kV.

According to an aspect, a method for treating an occlusion in a body lumen includes: providing a catheter with a central tube extending in a longitudinal direction from a distal region to a proximal region, an enclosure secured circumferentially around at least a portion of the distal region of the elongate tube, a first emitter assembly fixedly located on the central tube, a second emitter assembly fixedly located on the central tube, and a longitudinally translatable elongate member having a distal region movably located along the central lumen; inserting the catheter in the body lumen and locating the enclosure adjacent to the occlusion; filling the enclosure with a conductive fluid and anchoring the enclosure to a wall of the body lumen; moving the distal region of the elongate member to the first emitter assembly; and supplying power via the elongate member to the first emitter assembly and generating one or more shock waves at the first emitter assembly.

When power is supplied to the first emitter assembly, one or more shock waves may not be generated at the second emitter assembly.

The method may include moving the distal region of the elongate member to the second emitter assembly and supplying power via the elongate member to the second emitter assembly and generating one or more shock waves at the second emitter assembly.

The first emitter assembly may include one or more electrode pairs and moving the distal region of the elongate member to the first emitter assembly may include electrically connecting the distal region of the elongate member to the first emitter assembly.

### DESCRIPTION OF THE FIGURES

Illustrative aspects of the present disclosure are described in detail below with reference to the following drawing figures. It is intended that the embodiments and figures disclosed herein are to be considered illustrative rather than restrictive.

FIG. 1A illustrates an exemplary catheter having a movable array of emitters inside an angioplasty balloon, where the emitters are individually movable along the length of a catheter shaft, focusing on an exemplary wiring configuration, according to aspects of the present disclosure.

FIG. 1B illustrates an exemplary catheter having a movable array of emitters biased towards a proximal side of an enclosure, according to aspects of the disclosure.

FIG. 1C illustrates an exemplary catheter having an array of emitters that are movable as pairs along a length of an enclosure, according to aspects of the disclosure.

FIG. 1D illustrates an exemplary catheter having an array of emitters that are movable individually along a length of an enclosure, according to aspects of the disclosure.

FIG. 1E illustrates an exemplary catheter having a movable array of emitters that are biased toward a distal side of an enclosure, according to aspects of the disclosure.

FIG. 1F illustrates an exemplary catheter having a movable array of emitters with stops, according to aspects of the disclosure.

FIG. 1G illustrates a cross-sectional view of an exemplary catheter having a movable array of emitters, according to aspects of the disclosure.

FIG. 2A illustrates an exemplary catheter within a vascular structure, the catheter having a movable array of emitters inside an angioplasty balloon at a distal position, according to aspects of the present disclosure.

FIG. 2B illustrates an exemplary catheter within a vascular structure, the catheter having a movable array of emitters inside an angioplasty balloon at a central position, according to aspects of the present disclosure.

FIG. 2C illustrates an exemplary catheter within a vascular structure, the catheter having a movable array of emitters inside an angioplasty balloon at a proximal position, according to aspects of the present disclosure.

FIG. 3A illustrates a side view showing parts of an exemplary catheter having a plurality of movable emitter carriers, according to aspects of the disclosure.

FIG. 3B illustrates a cross-sectional view of the catheter of FIG. 4A.

FIG. 4A illustrates a first configuration of an exemplary catheter having a longitudinally translatable member, according to aspects of the disclosure.

FIG. 4B illustrates a second configuration of the catheter shown in FIG. 4A.

FIG. 5A illustrates a first configuration of an exemplary catheter having a longitudinally translatable member, according to aspects of the disclosure.

FIG. 5B illustrates a second configuration of the catheter shown in FIG. 5A.

FIG. 6 illustrates an exemplary flow chart for using a catheter with a movable array of emitters, according to aspects of the disclosure.

FIG. 7 illustrates another exemplary flow chart for using a catheter with a movable array of emitters, according to aspects of the disclosure.

FIG. 8 illustrates an exemplary catheter having a tapered enclosure, according to aspects of the disclosure.

FIG. 9 illustrates an exemplary catheter system with a rapid exchange port, according to aspects of the disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, and in U.S. Publication No. 2021/0085383, all of which are incorporated herein by reference. An example of a low-profile catheter with electrodes configured to be inserted after balloon expansion can be found in U.S. Pat. No. 10,357,264, which is incorporated herein by reference. Other catheter designs have improved the delivery of shock waves, for instance, by specific electrode construction and configuration thereby directing shock waves in a forward direction to break up tighter and harder-to-cross occlusions in vasculature. Examples of forward-firing catheter designs can be found in U.S. Pat. Nos. 10,966,737, 11,478,261, and 11,596,423 and U.S. Publication Nos. 2023/0107690 and 2023/0165598, all of which are incorporated herein by reference. U.S. Pat. No. 11,779,363, which is incorporated herein by reference, describes positioning shock wave emitters to promote constructive interference of acoustic waves.

As used herein, the term “electrode” refers to an electrically conducting element (typically made of a metal or alloy) that receives electrical current and subsequently releases the electrical current to another electrically conducting element. Accordingly, as used herein, an “electrode pair” refers to two electrodes that are positioned adjacent to each other such that an electrical current provided to one electrode will transmit across the gap (also referred to as a “spark gap”) between the two electrodes (e.g., between a first electrode and a second electrode, or vice versa, optionally with an insulation separating the two electrodes, and with the electricity passing through a conductive fluid or gas therebetween). Further, as used herein, an “emitter” refers to a structure that has one or more electrode pairs. Emitters can be singular, paired, or otherwise arranged together to be electrically connected as an emitter assembly. Shock waves can be generated at each electrode pair of an emitter. In some contexts, one or more electrode pairs, which can be positioned across one or more emitters, can also be referred to as an “electrode assembly”.

Typically, long calcified lesions in arteries and other vasculature present challenges for many interventional devices and users. A user (e.g., a physician) will assess the length and diameter of calcified lesions, then determine the diameter and length of the balloon catheter that they want to use during the procedure. To treat relatively long calcified lesions, a user may prefer to use a single long balloon to treat such lesions, selecting balloons having a length of from about one hundred to three hundred millimeters (100 mm-300 mm) and having a diameter of from two to thirty millimeters (2 mm-30 mm) when inflated.

In the context of current commercial IVL devices, the use of longer balloons presents a different catheter profile and thus different requirements to ensure adequate therapeutic function. In considering modifications to achieve therapeutic performance in a relatively long balloon, it is necessary to be mindful that there is a direct correlation between the number of emitters (given a fixed source of energy) and how effective IVL therapy is. In other words, as the number of emitters increases to accommodate relatively long balloons for long lesions, the energy available to satisfy the increased number of emitters diminishes and IVL efficacy may diminish as a result. This issue can be addressed in part by adding more channels to the IVL generator and system, although this solution results in a catheter that has more circuitry and wires, and thus a larger profile which may have more difficulty reaching smaller peripheral vasculature or crossing heavily occluded lesions. Current commercial IVL devices have two to five emitters within a balloon, where the emitters are adhesively bonded—and thus static in location—on the catheter shaft. Further, these devices have balloons with diameters from about twelve millimeters to sixty millimeters (12 mm-60 mm), where the working length of the balloon along the catheter is from one hundred ten millimeters to one hundred thirty-eight millimeters (110 mm-138 mm). This allows for a physician to go through a cycle of positioning, inflation, shock wave therapy, and deflation, moving the balloon over a catheter guidewire along the length of the lesion to treat the full length of the lesion. The emitters on these devices can pulse one at a time, two at a time, all at the same time, and/or pulse in an alternating or sequential pattern along the length of the catheter and balloon.

Described herein are catheters, balloons, and IVL circuits incorporating design elements that allow for treatment of relatively long calcified lesions, using an enclosure having an array of movable emitters. In such implementations, it is generally not necessary to go through an inflation and deflation cycle that requires repositioning the balloon for shock wave treatment as described above. Rather, the single balloon allows for movement of the emitters within the balloon to deliver shock wave treatment to target regions of a given long lesion. This can be accomplished with the long balloon slightly inflated or semi-inflated, depending on the calcium stricture within the artery or other vessel.

In some embodiments as illustrated, each of the emitters includes a cylindrical sheath (alternatively referred to as a ring or a band) mounted on and surrounding a movable emitter carrier. The cylindrical sheaths are formed of an electrically conductive material (e.g., a metal or an alloy) that accordingly forms a first electrode surface of an electrode pair. The sheath may include a cut-out region (e.g., a central circular hole, an arcuate cut-out on the edge of the sheath, etc.) that provides an unobstructed electrical path to a second electrically conductive material, specifically a conductive member (e.g., a copper wire, a flat coil, etc.) positioned underneath or within the movable emitter carrier, that thereby forms a second electrode surface of the electrode pair. In this configuration of emitter, the conductive portion of the sheath can be referred to as an outer electrode and the wiring can be referred to as an inner electrode. Electric current delivered across the emitters can jump across the space between the two electrode surfaces, also referred to as a “spark gap”, and generate a shock wave as described above. Either of the electrode surfaces can be the anode or the cathode, depending on the polarity of the pulse delivered across the emitters.

In many implementations, each emitter will have two electrode pairs, where current can travel from one electrode pair to the other electrode pair on the emitter by travelling across the electrically conductive material of the sheath. The electrode pairs on each emitter can be positioned to generate shock waves in directly opposite directions (i.e., arranged 180 degrees apart from each other around the catheter) or the electrode pairs can be positioned to generate shock waves in a convergent or biased direction (i.e., arranged at less than 180 degrees apart from each other around the catheter). In alternative implementations, an emitter can have three, four, five, or six electrode pairs arranged around the circumference of the emitter. The various emitters of the movable emitter array can be electrically connected to each other in a series or in parallel, on one or more electrical channels.

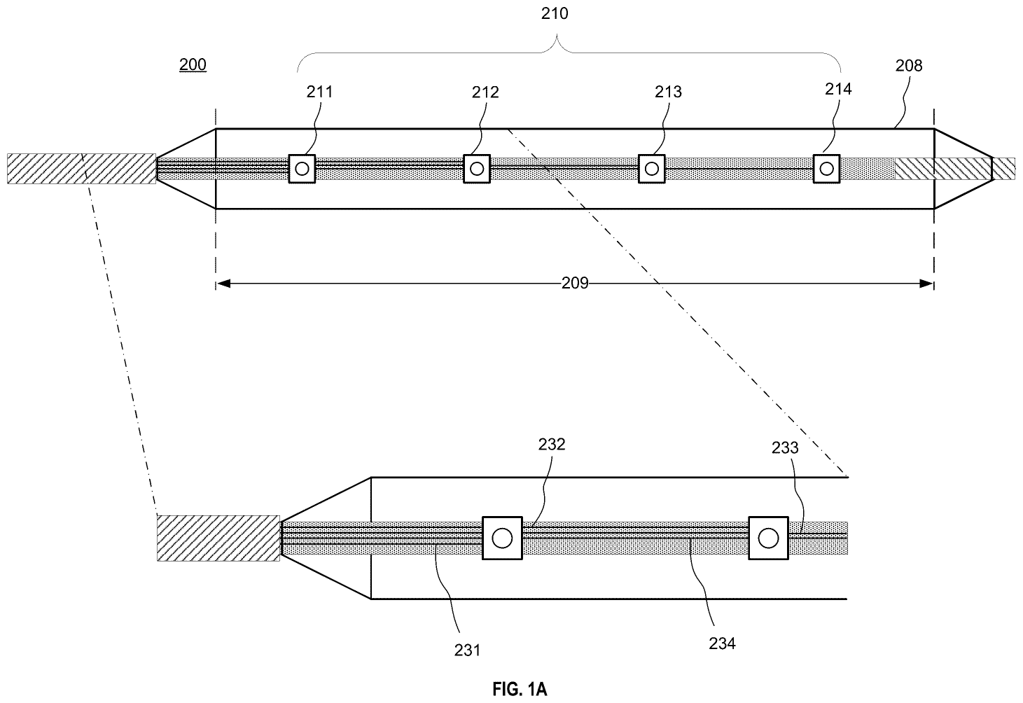

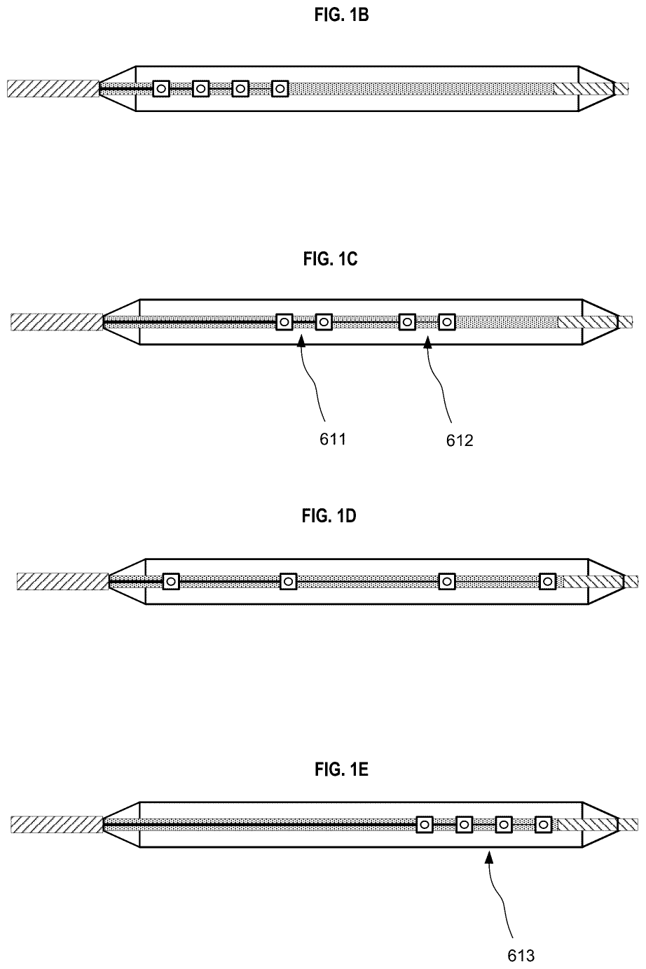

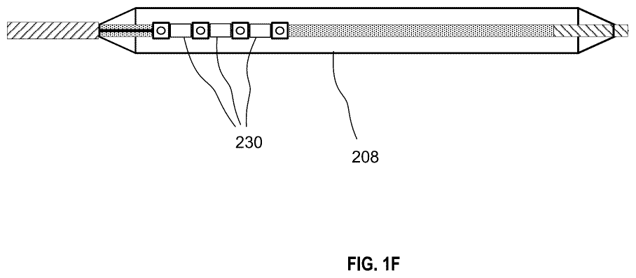

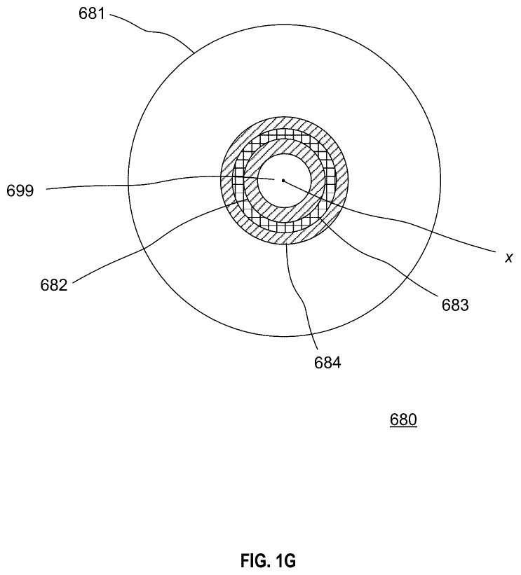

FIG. 1A illustrates catheter 200. As part of the catheter 200, a balloon 208 has a proximal end and a distal end, where the proximal end of the balloon 208 is attached to an outer balloon shaft and where the distal end of the balloon 208 is attached to an inner shaft. The balloon 208 may be attached at both locations such that the inner volume of the balloon 208 is sealed, for example with an adhesive, with a clamp, by making a heat seal, by making a pressure seal, or a combination thereof. At the proximal end, the balloon 208 is attached around the circumference of the balloon shaft such that a space between the balloon shaft and movable emitter carriers can be used as a channel for fluid to enter and inflate balloon 208 (and also to correspondingly egress and deflate balloon 208). In some embodiments, another type of sealed enclosure (e.g., a cap or a flexible polymer tube) may be used instead of an angioplasty balloon.

The working length of the balloon (e.g., working length 209) along the catheter can be from fifty millimeters to five hundred millimeters (50 mm-500 mm). The working length of the movable emitter carrier within the volume of the balloon can be up to the full length of the balloon. In some embodiments, a distance from the most proximal emitter to the most distal emitter may be up to the full working length of the balloon.

In some embodiments, on the movable emitter carrier, each emitter can be from three millimeters to twenty millimeters (3 mm-20 mm) distant from each other. The emitters can be electrically grouped (wired together) together on various channels of a circuit, where the emitters can be wired individually, as pairs, as triplets, or so on, and wired in series or in parallel. The power source supplying electricity to the emitters can supply power in the range of from about 1,000 V to about 15,000 V.

In various embodiments, a catheter having a movable emitter carrier having one or more shock wave emitters may include a relatively long enclosure (e.g., an angioplasty balloon). For example, the enclosure may have a working length that is 50%-200% longer than an end-to-end distance from a most proximal emitter to a most distal emitter. To employ such a device, the balloon may be advanced through a body lumen to a lesion. A user may then inflate the balloon with a fluid (e.g., saline) to a relatively low pressure (e.g., less than 5 atm) or until the working region of the balloon apposes (i.e., contacts) the lesion. Upon inflation, an emitter assembly mounted a movable emitter carrier may be advanced into the balloon volume to a first location (e.g., at a proximal location of the balloon) to treat the lesion or part of the lesion at the first location with shock wave therapy. The movable emitter carrier may then be farther advanced to a second location (e.g., at a more distal location than the first location) to treat a new lesion or a new region of the same lesion. In some embodiments, treatment may start at a distal region of the balloon or in a central region of the balloon. Advantageously, shock wave therapy may be customized for any particular lesion. For example, if there is a greater degree of calcification at a distal region of the balloon than at a proximal region of the balloon, more shock waves may be generated with the emitters at the more distal position than at the proximal position. By tuning the shock wave therapy in such a manner, unnecessary excess shock wave generation may be avoided, preserving the life of the device. Such devices are also less complicated in design than separately providing power (e.g., by electrical wires) to a large number of emitters located along a length of the angioplasty balloon.

FIG. 1A illustrates an exemplary catheter 200, according to one or more embodiments. The catheter 200 includes a movable array of emitters 210 inside an angioplasty balloon 208, where the emitters are individually movable along the length of a catheter shaft, focusing on an exemplary wiring configuration. The movable array of emitters 210 may include emitters 211, 212, 213, 214. In some examples, each emitter 211, 212, 213, 214 is individually movable within a working length 209 of balloon 208. For example, emitter 211 may be movable between a proximal end of the working length 209 to a location of emitter 212 relative to positions of the other emitters.

As more clearly seen in the enlarged image of FIG. 1A, the emitters 211, 212, 213, 214 are respectively connected by conductors (e.g., wires) 231, 232, 233, 234 that are electrically connected to a high voltage power supply at their proximal ends. Each of the emitters 211, 212, 213, 214 is also electrically connected to a common return wire (not shown) that is also connected to the high voltage power supply. In embodiments where each emitter is individually movable, each emitter is connected by a conductor to the power supply. In embodiments where emitters are movable as electrically connected pairs or groups, each pair or group is connected by one conductor to the power supply (as shown, e.g., in FIGS. 1C and 1E).

In some embodiments and as illustrated in FIG. 1F, one or more stops 230 may be provided between adjacent emitters to ensure that emitters are spaced from one another. In some embodiments, the stop 230 may have a proximal end to distal end length at least 1 mm. In some embodiments, the proximal end to distal end length of the stop 230 may be at least 2 mm. These stops may be translatable with an emitter (or emitter pair/grouping). In some examples, a stop may be provided at a proximal handle. For example, at the proximal handle, thumbwheel ratchets (or another kind of control) may be provided to individually control the translation of each emitter (or emitter pair/grouping) within the balloon 208 and be provided with stops to ensure that adjacent emitters are spaced from each other. Sufficient spacing between adjacent emitters (e.g., a spacing of at least 2 mm) may ensure optimal pressure output during shock wave therapy.

FIGS. 1B-1E illustrate exemplary catheters having a movable array of emitters inside an angioplasty balloon, where the emitters are movable along the length of a catheter shaft, further showing exemplary deployment configurations for the movable emitters within the angioplasty balloon.

FIG. 1B shows individually movable emitters configured to be biased toward the proximal end of the balloon for treating lesions at a proximal side of the balloon. Upon treatment at the proximal side of the balloon, one or more of the emitters may be moved distally to treat one or more lesions at more distal regions of the balloon. Advantageously, because the emitters are individually wired in these embodiments, shock waves do not need to be indiscriminately generated at all of the emitters in the balloon. In some embodiments, emitters may be connected at their proximal ends to different power outputs such that emitters are capable of generating more energetic or less energetic shock waves. Accordingly, an emitter (or emitters) connected to a higher power output may be positioned at a more stenosed lesion (or region of a lesion). Such energy distribution may be helpful to preserve device life.

FIG. 1C shows the emitters arranged as pairs next to each other, biased towards the center of the balloon. In this embodiment, emitters are configured to be moved in pairs (e.g., emitter pairs 611 and 612). Emitters of each emitter pair may be physically and electrically connected.

FIG. 1D illustrates another embodiment of individually movable emitters spread roughly equidistantly apart from each other along the full length of the balloon.

FIG. 1E illustrates another IVL catheter having movable emitters, according to one or more embodiments. Similar to some of the embodiments described above, this catheter includes a group of shock wave emitters 613 that is translatable as a group within a working region of a balloon. But in contrast, emitters 613 may not be retractable to outside of the balloon.

FIG. 1G illustrates a cross-sectional view of an IVL catheter 680 having emitters that are longitudinally movable (i.e., movable along a proximal-distal axis x), according to one or more embodiments. The cross-section is taken at a proximal region (proximal to any emitters) of a balloon 681, shown in an inflated state in FIG. 1G. Catheter 680 includes an inner shaft 682 that is tubular in structure and defines a guidewire lumen 699. A first movable emitter carrier 683 is located externally to the inner shaft 682. A second movable emitter carrier 684 is located externally to the first movable emitter carrier 683. The first movable emitter carrier 683 is translatable along the x-axis over inner shaft 682. The second movable emitter carrier 684 is translatable longitudinally over inner shaft 682 and first movable emitter carrier 683. In various embodiments, the two movable emitter carriers are independently movable for moving a first emitter assembly positioned on the first emitter carrier and a second emitter assembly positioned on the second emitter carrier (similar to the two grouped emitters shown in FIG. 1E). Each emitter carrier includes at its respective distal regions one or more shock wave emitters. Each emitter carrier additionally may include grooves or lumen for positioning energy guides (e.g., wires or optical fibers) that extend from a power source (e.g., a high voltage power supply or a laser) distally to one or more emitters. Although only two emitter carriers are shown in FIG. 1G, additional concentric emitter carriers may be included, and, in some embodiments, an IVL catheter has three, four, five, or more independently movable emitter assemblies (where each emitter assembly includes one or more shock wave emitters).

In some embodiments, the most inner movable emitter carrier is movable to the most distal region of the balloon. For example, first emitter carrier 683 may be translated to a most distal region of the balloon. Second emitter carrier 684 may be translated to a location of the first emitter assembly, but not more distally than the first emitter assembly. In other words, the first emitter carrier moves a more distal emitter assembly and the second emitter carrier moves a more proximal emitter assembly.

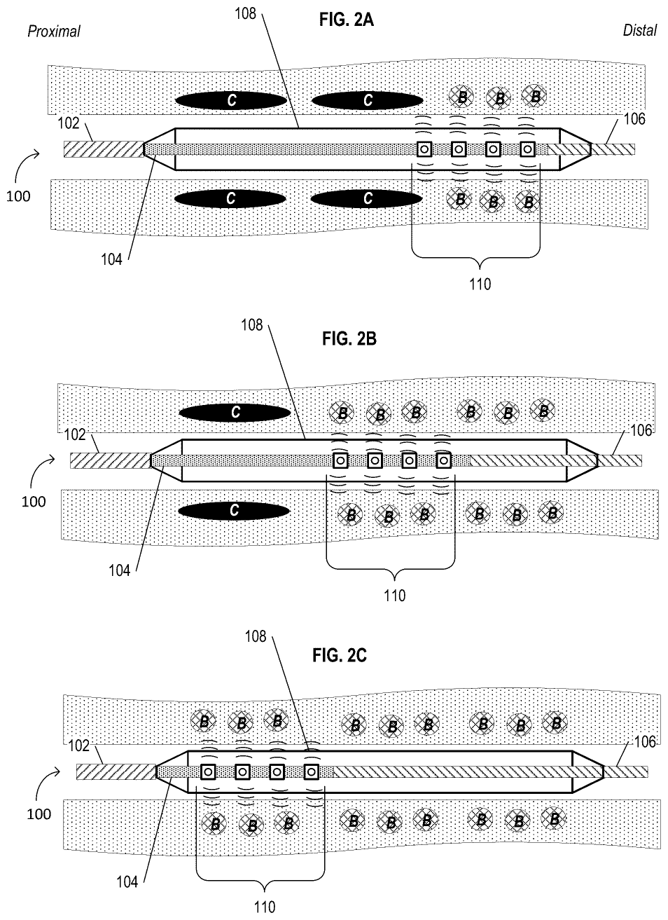

FIGS. 2A-2C illustrate an exemplary catheter 100 within a vascular structure, the catheter having a movable array of emitters inside an angioplasty balloon shown at a distal position in FIG. 2A, at a central position in FIG. 2B, and at a proximal position in FIG. 2C. As illustrated, calcified regions of vascular tissue are indicated with a “C” and regions of vascular tissue where the calcification has been subjected to shock waves and broken apart are indicated with a “B”. Starting with FIG. 2A, the inflated balloon 108 extends along the full length of the vascular tissue having calcification. The movable emitter carrier 104 extends along the length of inner shaft 106 to the distal end of balloon 108, with movable emitter array 110 positioned at the distal end of movable emitter carrier 104. Accordingly, the movable emitter array 110 is adjacent to the calcification at the distal end of the subject lesion in the vascular tissue. As illustrated, the emitters of movable emitter array 110 generate shock waves that break apart the calcification of the distal portion of the lesion.

Moving to FIG. 2B, the movable emitter carrier 104 has been pulled back in a distal direction along the length of catheter 100 such that movable emitter array 110 is adjacent to calcification in the middle region of the subject lesion in the vascular tissue. As illustrated, the emitters of movable emitter array 110 generate shock waves that break apart the calcification in this middle portion of the lesion. It should be appreciated that the middle region of a long, calcified lesion may require repositioning of the movable emitter array 110 multiple times to deliver treatment to the complete length of middle regions of the calcified lesion.

Finally, moving to FIG. 2C, the movable emitter carrier 104 has been pulled back in a distal direction along the length of catheter 100 such that movable emitter array 110 is adjacent to calcification at the proximal of the subject lesion in the vascular tissue. As illustrated, the emitters of movable emitter array 110 generate shock waves that break apart the calcification of the proximal portion of the lesion.

It should be appreciated that more than one cycle of shock wave generation can be used to break down calcification in any given region of the calcified tissue, and that different regions of calcified tissue may need relatively more or less shock wave treatment as compared to each other. It can be further appreciated that the sequence of translating the movable emitter array 110 can proceed from distal to proximal as described in FIGS. 2A-2C, but can alternatively proceed in a proximal-to-distal sequence, in a medial-to-proximal, or a medical-to-distal sequence. Further, the movable emitter array 110 can be moved to repeat treatment at locations along the calcified tissue two or more times.

The materials that can be used for components such as the outer shaft 102, movable emitter carrier 104, and inner shaft 106 can be extruded or molded polymers, or functional equivalents that can be safely used in a patient body. Such materials may include polyether block amide, polytetrafluoroethylene, nylon, or other polymers.

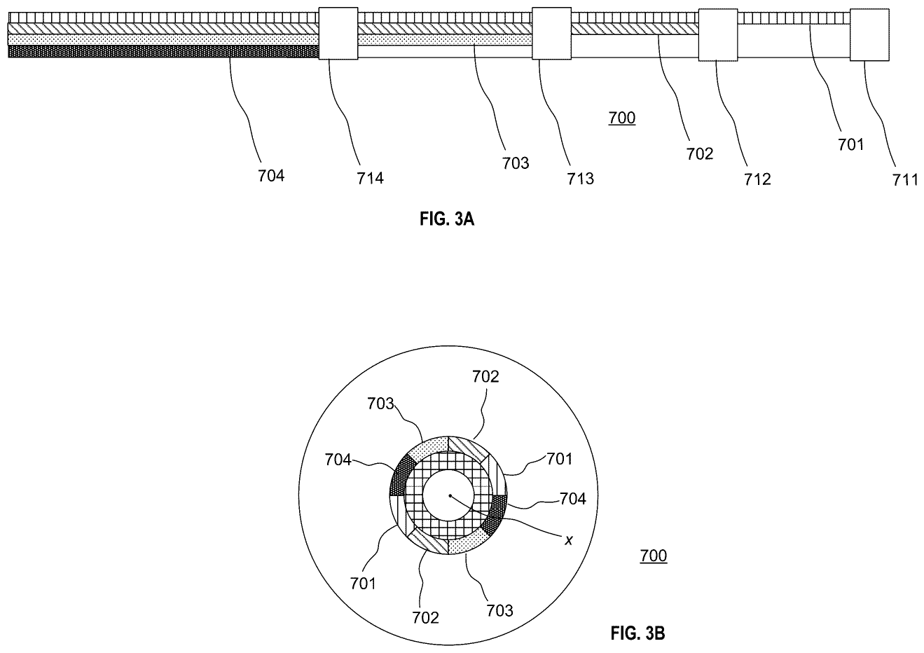

FIGS. 3A and 3B illustrate part of an IVL catheter 700 having independently movable emitters, according to one or more embodiments. As shown in the side view schematic of FIG. 3A, catheter 700 includes first, second, third, and fourth movable emitter carriers 701-704. First emitter carrier 701 extends to first emitter 711, second emitter carrier 702 extends to 712, third emitter carrier 703 extends to emitter 713, and fourth emitter carrier 704 extends to emitter 714. Each of the emitter carriers 701-704 is independently translatable relative to the other emitter carriers along a direction parallel to longitudinal axis x. Rather than the emitter carriers being concentrically stacked on each other (as in the example shown in FIG. 1G), each of the emitter carriers of catheter 700 includes one or more legs that are circumferentially arranged next to each other, as shown in the cross-section of FIG. 1B. Each of the emitter carriers 701-704 extends from a proximal handle or hub distally to an emitter assembly. In the example in FIG. 1A, each emitter carrier is shown to extend to a single emitter, but in other embodiments, each emitter carrier may extend to an emitter assembly having more than one emitter that are electrically connected to each other. Having such a slotted emitter carrier design may help to reduce the catheter's profile and make the device more navigable through narrow body lumens.

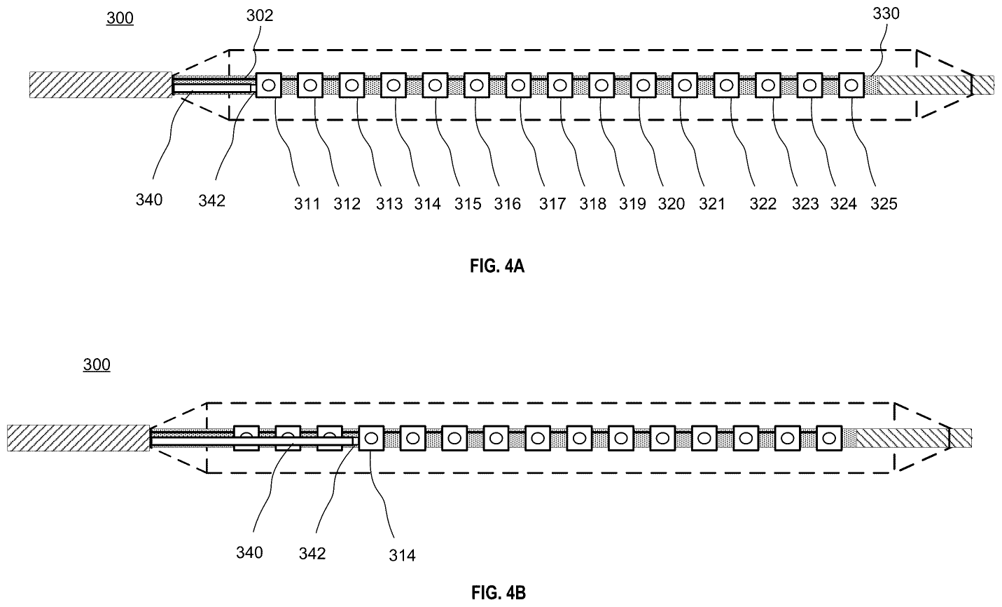

FIGS. 4A and 4B illustrate an exemplary catheter 300, according to one or more aspects of the disclosure. The catheter 300 includes a plurality of shock wave emitters 311-325 located along a central elongate member 330 and a longitudinally translatable member 340 electrically connected at its proximal end to a power supply (not shown). The longitudinally translatable member 340 is shown as being external to the emitters in the figures; in some embodiments, the translatable member 340 may be translatable within a groove or lumen of the central elongate member 330, which may help to keep the translatable member 340 aligned during translation and positioning. Each of the shock wave emitters 311-325 includes an electrode pair having a spark gap, and a first electrode of the electrode pair is electrically connected to a return wire 302 that is electrically connected to the power source. The longitudinally translatable member 340 may include an electrically conductive region 342. FIG. 4A illustrates a configuration where the translatable member 340 is positioned such that the electrically conductive region 342 is in electrical contact with a second electrode of the electrode pair of emitter 311. In this configuration, a high voltage pulse from a power source may be delivered via the translatable member 340 across the electrode pair of emitter 311, generating a shock wave. FIG. 4B illustrates another configuration of the catheter 300 where the longitudinally translatable member 340 is advanced more distally than in the configuration shown in FIG. 4A such that the translatable member 340 is electrically connected to emitter 314. In this configuration, a high voltage pulse is delivered to emitter 314 resulting in shock wave generation at emitter 314.

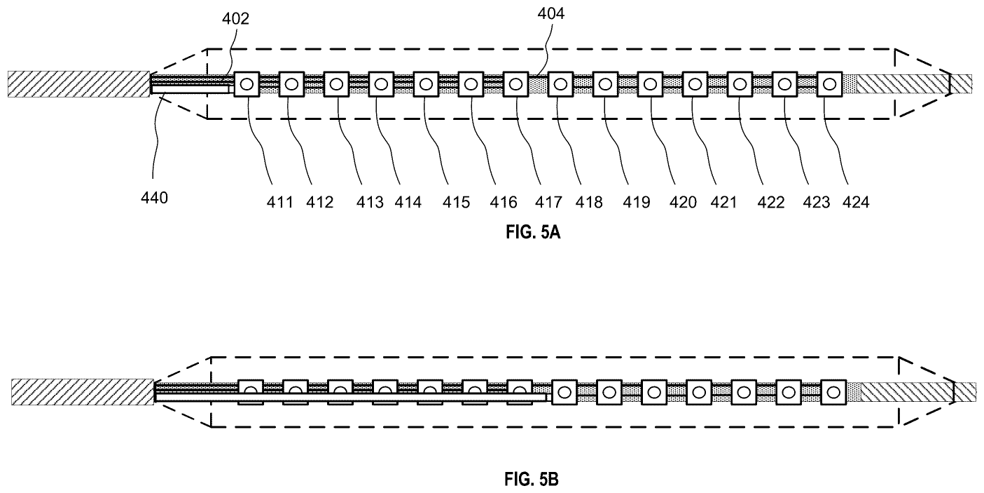

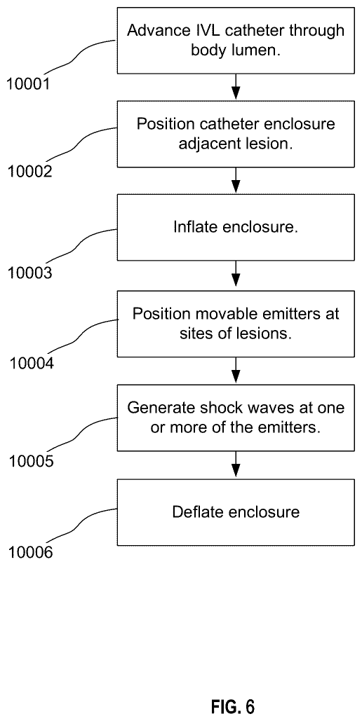

FIGS. 5A and 5B illustrate an exemplary catheter 400, according to one or more aspects of the disclosure. The catheter 400 includes a plurality of shock wave emitters 411-424 and a longitudinally translatable member 440 electrically connected to a power supply (not shown). Each emitter may be electrically connected to one or more other emitters such that when the translatable member 440 is electrically connected to that emitter, shock waves are generated at each of the connected emitters. FIG. 5A illustrates a first configuration where the translatable member 440 is electrically connected to shock wave emitters 411-417, which are connected in series to each other and electrically connected to a first return wire 402. When a high voltage pulse is supplied to shock wave emitters 411-417, a shock wave is generated at each of the emitters 411-417. FIG. 5B illustrates a second configuration where the translatable member 440 is electrically connected to shock wave emitters 418-424, which are connected in series to each other and electrically connected to a second return wire 404. When a high voltage pulse is supplied to shock wave emitters 418-424, a shock wave is generated at each of the emitters 418-424. In other embodiments, a longitudinally translatable member may supply power to pairs of emitters. An emitter positioned adjacent to a thicker part of the lesion may be supplied with a relatively higher energy (e.g., a higher voltage pulse) than an emitter positioned adjacent to a thinner part of the lesion. Additionally, or alternatively, a greater number of emitters may be positioned adjacent to the thicker part of the lesion than the thinner part. Steps 10004 and 10005 may be repeated to treat other additional lesion sites. As with other IVL treatment methods, the enclosure may be deflated and inflated with saline (or other conductive fluid) to remove any accumulated gas bubbles within the enclosure. After treatment, at step 10006, the enclosure may be deflated and the IVL catheter withdrawn or moved to a different lesion.

In one or more embodiments, positioning of the emitters may be imaged by an imaging method, such as x-ray fluoroscopy. Accordingly, in some embodiments, each of the movable emitters (or individual emitter assemblies) may include a radiopaque marker. In some embodiments, positions of the emitters may be indicated by indicia at a proximal end of the catheter.

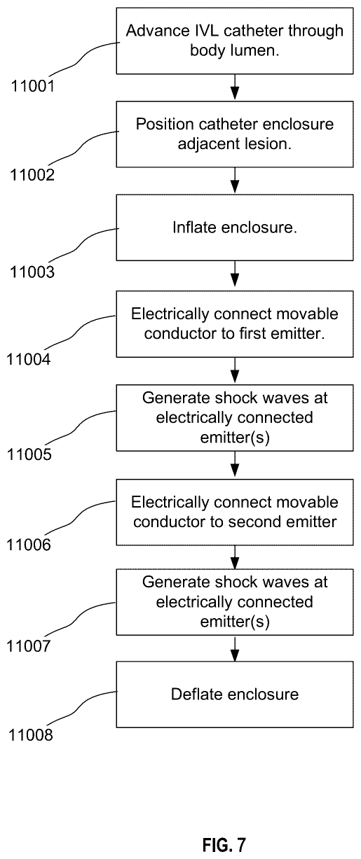

FIG. 7 is a flow chart of a method for using an IVL catheter having a movable conducting member, according to one or more embodiments. The catheter in these embodiments is similar to those shown in FIGS. 4A, 4B, 5A, and 5B and includes a plurality of emitters (or emitter assemblies) that are not electrically connected to a power supply. At step 11001, the catheter is advanced through a body lumen. At step 11002, an enclosure of the catheter is positioned adjacent to a lesion of the body lumen. At step 11003, the enclosure is inflated with a fluid to a relatively low pressure (e.g., less than 5 atm) such that the enclosure is in apposition to the lesion and vessel wall. In one or more embodiments, the enclosure is made of a semi-compliant material such that it can conform to the geometry of the lesion when inflated to the relatively low pressure. At step 11004, a conducting region of a movable conductor is moved to and electrically connected to a first emitter of the plurality of emitters. In some embodiments, the movable conductor is withdrawn in a location proximal to the enclosure during delivery and then inserted into the balloon at step 11004. This helps to reduce the catheter's profile during delivery and also improves flexibility at the distal region of the catheter. At step 11005, a high voltage pulse is delivered to the first emitter to generate a shock wave at the electrically connected emitter. In some embodiments, the first emitter is electrically connected (e.g., in series or in parallel) to one or more emitters) such that when the high voltage pulse is delivered, a shock wave is generated at each of the connected emitters. At step 11006, the conducting member is moved to a second emitter (or second set of electrically connected emitters) at a different location within the enclosure. At step 11007, shock waves are generated at the one or more electrically connected emitters. Steps 11006 and 11007 may be repeated at different emitters (or emitter assemblies) to generate shock waves at other regions of the enclosure. Between rounds of shock wave generation, the enclosure may be deflated and inflated to remove any accumulated gas bubbles. At step 11008, upon completion of shock wave therapy at the lesion being treated, the enclosure is deflated.

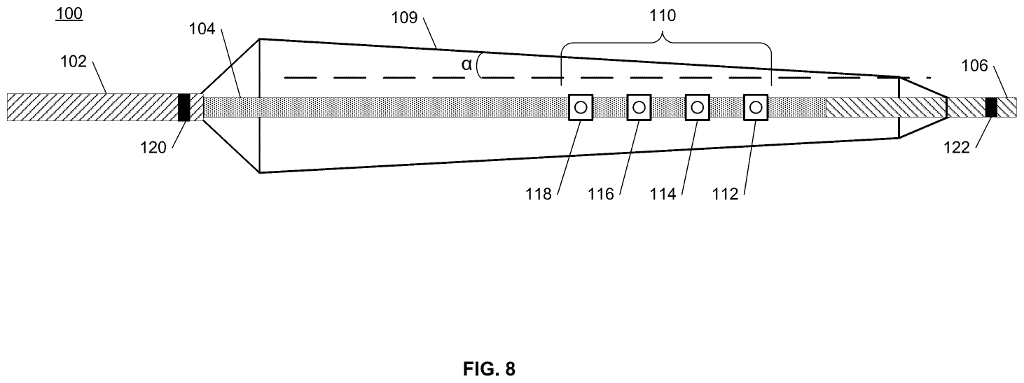

FIG. 8 illustrates an exemplary catheter having a movable array of emitters 110 inside a tapered angioplasty balloon 109 at a distal position. The use of a tapered angioplasty balloon 109 where the distal end is narrower than the proximal end can provide for improved access to vasculature that may be partially obstructed with plaque (e.g., a chronic total occlusion (CTO)), where the narrow end of the tapered angioplasty balloon 109 can function as a wedge to push into and through the plaque. In such situations with the tapered angioplasty balloon 109 proximate to or in contact with a device-facing surface of vascular plaque, the movable emitter carrier 104 may be positioned in a distal location and shock waves can be generated to break down the calcification of the surrounding lesion at the entry point of that vascular plaque. The relaxing or loosening of the vasculature at that location can then allow for the tapered angioplasty balloon 109 to be pushed further along the vessel and through the vascular plaque, to ultimately allow for shock waves to be generated along the whole length of the calcified lesion. A tapered angioplasty balloon may also be implemented in body lumens that naturally taper. In some embodiments, a tapered angioplasty balloon has a tapered working region with a tapering angle (e.g., angle α) up to 20 degrees.

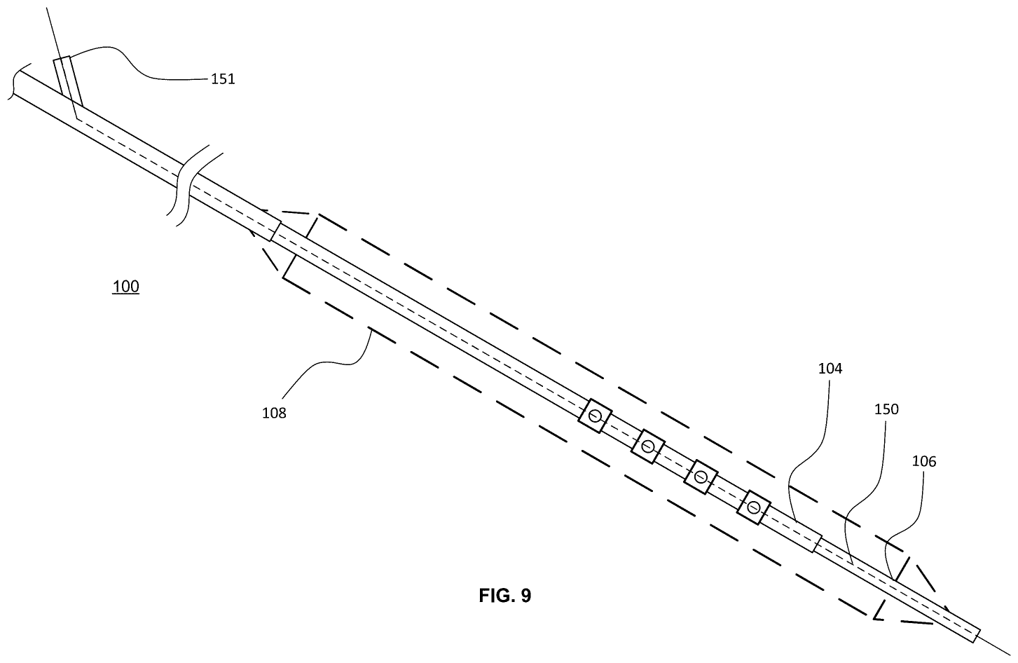

In some examples described above, the catheter may be configured to be advanced into the body lumen over a guide wire, generally referred to as an “over-the-wire” (OTW) arrangement. In alternative implementations, as illustrated in FIG. 9, the catheter may be arranged as part of a “rapid exchange” (Rx) configuration where instruments are swapped in and out of a patient through a larger lumen during a procedure. In this FIG. 9, guidewire 150 and inner shaft 106 are included, and this includes a guidewire lumen. The guidewire 150 may exit the inner shaft 106 at a port 151 that is distal of a proximal end of the catheter (not shown) and proximal of the distal end of balloon 108. The emitter carrier 104 moves back and forth on the inner shaft 106, which is static. The inner shaft 106 may be bonded to a proximal tip of the balloon to keep the Rx guidewire stable during treatment. The catheter 100 may include a fluid lumen that extends through outer shaft 102 to a proximal end of the catheter 100 for delivering fluid to and moving fluid from the interior of balloon 108. It should be readily understood that such a fluid lumen can be used in OTW configurations as well as Rx configurations of the catheter 100.

It should be noted that the elements and features of the example catheters illustrated herein may be rearranged, recombined, and modified without departing from the present invention. For instance, the figures illustrate example electrode assemblies, the present disclosure is intended to include catheters having a variety of electrode configurations, and the number, placement, and spacing of the emitters and electrode pairs can modified without departing from the subject invention.

Although the electrode assemblies and catheter devices described herein have been discussed primarily in the context of treating occlusions and lesions in coronary vasculature, the electrode assemblies and catheters herein can be used for a variety of occlusions and peripheral vasculature (e.g., above-the-knee, below-the-knee, iliac, carotid, etc.), and other anatomy can be treated using IVL. For further examples, implementations of the embodiments disclosed herein may be used for treating soft tissues, such as cancer and tumors (i.e., non-thermal ablation methods), blood clots, fibroids, cysts, organs, scar and fibrotic tissue removal, multi-morphology tissue, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

Further, while the emitters disclosed in examples herein have a construction typically having two electrode pairs on each emitter, it is contemplated to also include emitters having three electrode pairs (for example, circumferentially separated from each other by 120 degrees), four electrode pairs (for example, circumferentially separated from each other by 90 degrees), five electrode pairs (for example, circumferentially separated from each other by 72 degrees), six electrode pairs (for example, circumferentially separated from each other by 60 degrees), and so on. There may be physical limitations to the construction of such emitter assemblies relating to the size and arrangement of wiring, the ability to deliver sufficient power, the erosion profile of electrodes, and so on as it is within the scope of the present disclosure that such emitters may be successfully developed with improvements in manufacturing capabilities.

Furthermore, numerical designators such as “first”, “second”, “third”, “fourth”, etc. are merely descriptive and do not necessarily indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It will be understood that the foregoing is only illustrative of the principles of the invention, and that various modifications, alterations, and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except by the appended claims.

1. A catheter for treating an occlusion in a body lumen, the catheter comprising:
an elongate tube extending in a longitudinal direction from a distal region to a proximal region;
a flexible enclosure at least partially secured to the distal region of the elongate tube;
a first shock wave emitter located along the elongate tube; and
a second shock wave emitter located along the elongate tube and translatable in the longitudinal direction relative to the first shock wave emitter.

2. The catheter of claim 1, wherein the flexible enclosure has a working length d, and a center-to-center distance between the first shock wave emitter and the second shock wave emitter is adjustable between 2 mm and d.

3. The catheter of claim 2, wherein the first shock wave emitter comprises a first pair of shock wave emitters and the second shock wave emitter comprises a second pair of shock wave emitters that is translatable as a pair relative to the first pair of shock wave emitters.

4. The catheter of claim 1, wherein the first shock wave emitter comprises a first plurality of shock wave emitters and the second shock wave emitter comprises a second plurality of shock wave emitters that is translatable as a group relative to the first plurality of shock wave emitters.

5. The catheter of claim 1, further comprising a third shock wave emitter that is independently translatable relative to the first and second shock wave emitters.

6. The catheter of claim 5, further comprising a safety stop fixedly located on the elongate tube and configured to space the first and second shock wave emitters by a center to center distance no less than 2 mm.

7. The catheter of claim 1, wherein the first shock wave emitter is translatable in the longitudinal direction.

8. The catheter of claim 1, further comprising a proximal handle that is configured to control movement of at least one of the first shock wave emitter and the second shock wave emitter.

9. The catheter of claim 8, wherein the proximal handle comprises a first thumbwheel ratchet for controlling movement of the first shock wave emitter and a second thumbwheel ratchet for controlling movement of the second shock wave emitter.

10. The catheter of claim 1, wherein the flexible enclosure is an angioplasty balloon having a working length of at least 50 mm.

11. The catheter of claim 1, wherein each of the first and second shock wave emitters comprises one or more electrode pairs and each of the one or more electrode pairs comprises an outer electrode and an inner electrode.

12. A catheter for treating an occlusion in a body lumen, the catheter comprising:
an elongate tube extending in a longitudinal direction from a distal region to a proximal region;
an enclosure secured circumferentially around at least a portion of the distal region of the elongate tube;
a proximal emitter assembly fixedly located on the elongate tube and inside the enclosure;
a distal emitter assembly fixedly located on the elongate tube and inside the enclosure; and
a longitudinally translatable elongate member having a distal region movably located along the elongate tube, the elongate member configured to, in a proximal configuration, supply power to the proximal emitter assembly and, in a distal configuration, supply power to the distal emitter assembly.

13. The catheter of claim 12, wherein:
the proximal emitter assembly comprises one or more electrically connected proximal electrode pairs including a first proximal electrode that is electrically connected to a power supply, and, in the proximal configuration, the longitudinally translatable elongate member is electrically connected to a second proximal electrode of the proximal electrode pairs, such that when a voltage pulse is applied from the power supply to the proximal emitter assembly, each of the one or more proximal electrode pairs generates a shock wave; and
the distal emitter assembly comprises one or more electrically connected distal electrode pairs including a first distal electrode that is electrically connected to the power supply, and, in the distal configuration, the longitudinally translatable elongate member is electrically connected to a second distal electrode of the distal electrode pairs, such that when a voltage pulse is applied from the power supply to the distal emitter assembly, each of the one or more distal electrode pairs generates a shock wave.

14. The catheter of claim 13, wherein the one or more proximal electrode pairs and one or more distal electrode pairs each comprises an inner electrode and an outer electrode made of a conductive sheath.

15. The catheter of claim 12, wherein, in the proximal configuration, shock waves are generated at the proximal emitter assembly and not at the distal emitter assembly, and, in the distal configuration, shock waves are generated at the distal emitter assembly and not at the proximal emitter assembly.

16. The catheter of claim 12, wherein the distal region of the translatable elongate member includes a radiopaque marker.

17. The catheter of claim 12, further comprising a proximal handle to control movement of the elongate member between the proximal configuration to the distal configuration.

18. A method for treating an occlusion in a body lumen comprising:
providing a catheter including:
a central tube extending from a proximal region to a distal region and defining a longitudinal direction and having a central lumen,
an enclosure sealed to and surrounding at least a portion of the distal region of the central tube, and
a shock wave emitter assembly including a first shock wave emitter and a second shock wave emitter located along the central tube within the enclosure, the first and second shock wave emitters movable in the longitudinal direction relative to the other;
inserting the catheter into the body lumen and positioning the enclosure adjacent the occlusion;
filling the enclosure with a conductive fluid and anchoring the enclosure to a wall of the body lumen;
moving the shock wave emitter assembly 1 mm or less away in the longitudinal direction from the occlusion; and
generating one or more shock waves from the shock wave emitter assembly.

19. The method of claim 18, further comprising imaging the body lumen with one or more of x-ray fluorescence, intravascular ultrasound, and optical coherence tomography,
wherein the shock wave emitter assembly includes an imaging marker.

20. The method of claim 18, wherein each shock wave emitter comprises an electrode pair and generating the one or more shock waves comprises applying a high voltage pulse from a power source.

21. The method of claim 20, wherein the high voltage pulse includes a voltage between 1 kV and 15 kV.

22. A method for treating an occlusion in a body lumen comprising:
providing a catheter including:
a central tube extending in a longitudinal direction from a distal region to a proximal region,
an enclosure secured circumferentially around at least a portion of the distal region of the central tube,
a first emitter assembly fixedly located on the central tube,
a second emitter assembly fixedly located on the central tube, and
a longitudinally translatable elongate member having a distal region movably located along the central tube;
inserting the catheter in the body lumen and locating the enclosure adjacent to the occlusion;
filling the enclosure with a conductive fluid and anchoring the enclosure to a wall of the body lumen;
moving the distal region of the elongate member to the first emitter assembly; and
supplying power via the elongate member to the first emitter assembly and generating one or more shock waves at the first emitter assembly.

23. The method of claim 22, wherein when power is supplied to the first emitter assembly, one or more shock waves are not generated at the second emitter assembly.

24. The method of claim 22, further comprising moving the distal region of the elongate member to the second emitter assembly and supplying power via the elongate member to the second emitter assembly and generating one or more shock waves at the second emitter assembly.

25. The method of claim 22, wherein the first emitter assembly comprises one or more electrode pairs and moving the distal region of the elongate member to the first emitter assembly comprises electrically connecting the distal region of the elongate member to the first emitter assembly.
